(19) United States
(12) Patent Application Publication
(10) Pub. No.: US 2005/0203384 A1
(43) Pub. Date: Sep. 15, 2005

(54) Computer assisted system and method for minimal invasive hip, uni knee and total knee replacement

(76) Inventors: Marwan Sati, Mississauga (CA); Haniel Croitoru, Toronto (CA); Peter Tate, Georgetown (CA); Liqun Fu, Mississauga (CA)

Correspondence Address: Gowling Lafleur Henderson LLP, Suite 4900, Commerce Court West, Toronto, ON, M5L 1J3, CA

(21) Appl. No.: 11/016,878

(22) Filed: Dec. 21, 2004

Related U.S. Application Data
(63) Continuation of application No. PCT/CA03/00947, filed on Jun. 23, 2003.
(60) Provisional application No. 60/390,188, filed on Jun. 21, 2002.

(51) Int. Cl. A61B 5/05

(52) U.S. Cl. 600/426

(57) ABSTRACT

As a general overview, the system 10 is used to assist the surgeon in performing an operation by acquiring and displaying an image of the patent. Subsequent movement of the patient and instruments is tracked and displayed on the image. Images of a selection of implants are stored by the system and may be called to be superimposed on the image. The surgical procedures may be planned using the images of the patient and instruments and implants and stored as a series of sequential tasks referred to defined datums, such as inclination or position. Gestures of the surgeon may be used in the planning stage to call the image of the instruments and in the procedure to increment the planned tasks.

This application claims the benefit of U.S. Provisional Patent Application No. 60/390,188, entitled “COMPUTER ASSISTED SYSTEM AND METHOD FOR MINIMAL INVASIVE HIP, UNI KNEE AND TOTAL KNEE REPLACEMENT”, filed on Jun. 21, 2002.

### BACKGROUND OF THE INVENTION

Field of the Invention

The present invention relates to a method and system for computer assisted medical surgery procedures, more specifically, the invention relates to a system which aids a surgeon in accurately positioning surgical instruments for performing surgical procedures, and also relates to reducing user interaction with the system for minimal invasive surgery.

Many surgical procedures, particularly in the fields of orthopedic surgery and neurosurgery, involve the careful placement and manipulation of probes, cutting tools, drills and saws amongst a variety of surgical instruments. Computer-based surgical planning has been investigated by many researchers over the past decade and the promise of the technology is to provide better surgical results (with fewer procedures), decreased time in the operating room, lower resulting risk to the patient (increased precision of technique, decreased infection risk), and a lower cost. In image-guided surgery the vision of reality is enhanced using information from CT, MR and other medical imaging data. Certain instruments can be guided by these patient specific images if the patient's position on the operating table is aligned to this data.

Preoperative 3D imaging may help to stratify patients into groups suitable for a minimally invasive approach or requiring open surgery. The objectives include the most accurate prediction possible, including the size and position of the prosthesis, the compensation of existing differences in leg lengths, recognizing possible intraoperative particularities of the intervention, reducing the operating time and the potential for unforeseen complications.

Traditional surgical planning involves overlay of 2D templates onto planar X-ray images, however this process is sensitive to errors in planar X-ray acquisition and magnification. Precise 3D models of implants superposed onto intra-operative calibrated fluoro is an improvement over current methods, however interpretation of these 3D models is not intuitive.

In the case of X-ray imaging (fluoroscopy or CT scan), the surgical staff are required wear protective clothing, such as lead aprons during the procedure. Also, the imaging device must be present during the course of the surgery in case the patient's orientation is changed. This can be cumbersome and undesirable given the space requirements for such equipment, such as magnetic resonance imaging, X-ray imaging machine or ultrasound machine. Therefore, in such circumstances it is desirable to maintain the patient in a fixed position through the course of the surgical operation, which can prove to be very difficult. Therefore, a surgeon has to be present for image acquisition and landmark identification.

Image-guided surgery permits acquiring images of a patient whilst the surgery is taking place, align these images with high resolution 3D scans of the patient acquired preoperatively and to merge intraoperative images from multiple imaging modalities. Intraoperative MR images are acquired during surgery for the purpose of guiding the actions of the surgeon. The most valuable additional information from intraoperative MR is the ability for the surgeon to see beneath the surface of structures, enabling visualization of what is underneath what the surgeon can see directly.

The advantages of 2D operation planning include simple routine diagnostics, as the X-ray is in 2 planes, simple data analysis, simple comparison/quality control on postoperative X-ray, and more beneficial cost-benefit relation. However, 2D operation planning module has the several drawbacks, it lacks capability of spatially imaging of anatomic structures, and implant size can only be determined by using standardized X-ray technology and has no coupling to navigation. The advantages of 3D include precise imaging of anatomical structures, precise determination of implant size, movement analysis of the joint possible, and coupling with navigation. However, 3D provides for more expensive diagnostics, as it involves X-ray imaging and CT/MRI imaging. Also, CT data analysis is time consuming and costly, and there is no routine comparison of 3D planning and OP result (post-op. CT on routine.

### SUMMARY OF THE INVENTION

In one of its aspects there is provided a computer-implemented method for enhancing interaction between a user and a surgical computer assisted system, the method includes the steps of tracking a user's hand gestures with respect to a reference point; registering a plurality of gesturally-based hand gestures and storing said gestures on a computer-readable medium; associating each of said plurality of gesturally-based hand gestures with a desired action; detecting a desired action by referencing said user's hand gestures stored on said computer-readable medium; and performing the desired action.

In another one of its aspects there is provided a computer-implemented method for enhancing interaction between a user and a surgical computer assisted system, the method having the steps of: determining information for a surgical procedure from the orientation of a medical image whereby accuracy of said information is improved. The orientation of the medical image is obtained by tracking of the imaging device or by tracking of a fiducial object visible in the image.

In another one of its aspects there is provided a method for a computer assisted surgery system, the method includes the steps of using 3D implant and instrument geometric models in combination with registered medical images, generating 2D projections of that instrument and/or implant, updating the 2D projection dynamically in real-time as the implant/instrument is moved about in 3D space. Advantageously, the dynamic 2D projection is more intuitive and provides ease of use a user.

In yet another aspect of the invention, there is provided a method for a computer assisted surgery system, the method having the steps of displaying a magnified virtual representation of a target instrument or implant size while smaller instruments or implants are being used.

### BRIEF DESCRIPTION OF THE DRAWINGS

These and other features of the preferred embodiments of the invention will become more apparent in the following detailed description in which reference is made to the appended drawings wherein:

FIG. 1 is a schematic representation of a computer assisted surgery system;

FIG. 2 is a block diagram of a computing device used in the system of FIG. 1;

FIG. 3 is a set of instruments for use with the system of FIG. 1;

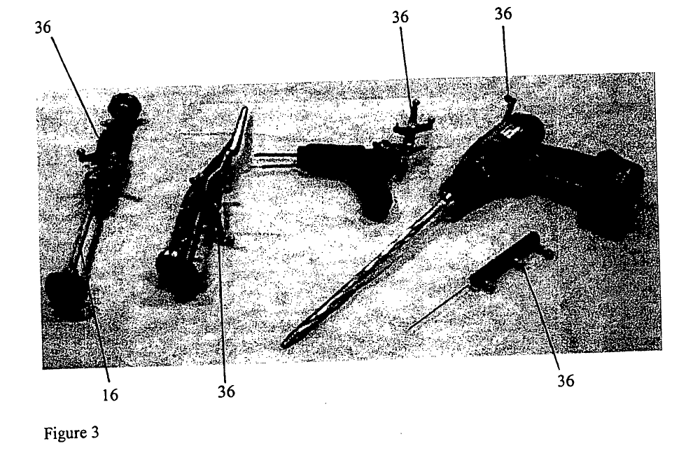

FIG. 4 is patient tracker for minimal invasive surgery;

FIG. 5 is a flow chart showing the sequential steps of using the system of FIG. 1.

FIG. 6 shows examples of landmarks defining a pelvic coordinate system;

FIG. 7 shows a way of calculating an anteversion or inclination angle;

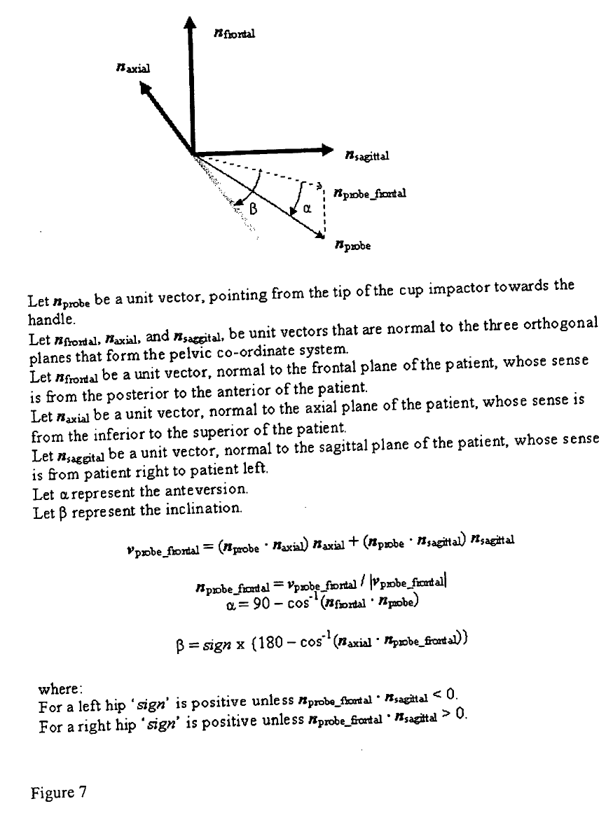

FIG. 8 shows a virtual representation of a reamer;

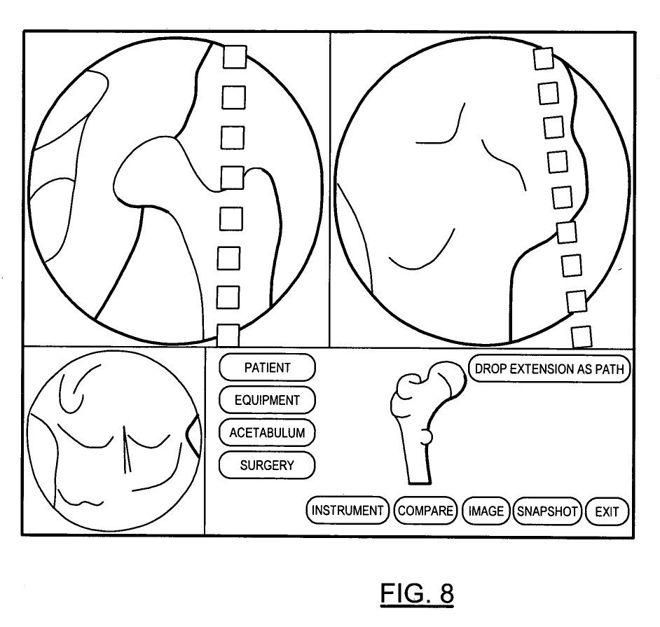

FIG. 9 shows a femoral anteversion;

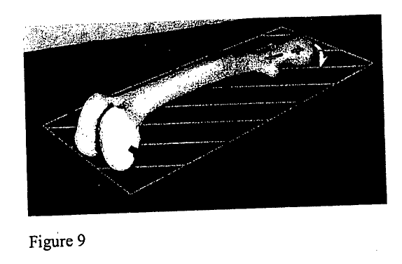

FIG. 10 shows guidance of a femoral stem length and an anteversion angle; and

FIG. 11 is a 2D projection of femoral stem model.

### DESCRIPTION OF THE PREFERRED EMBODIMENTS

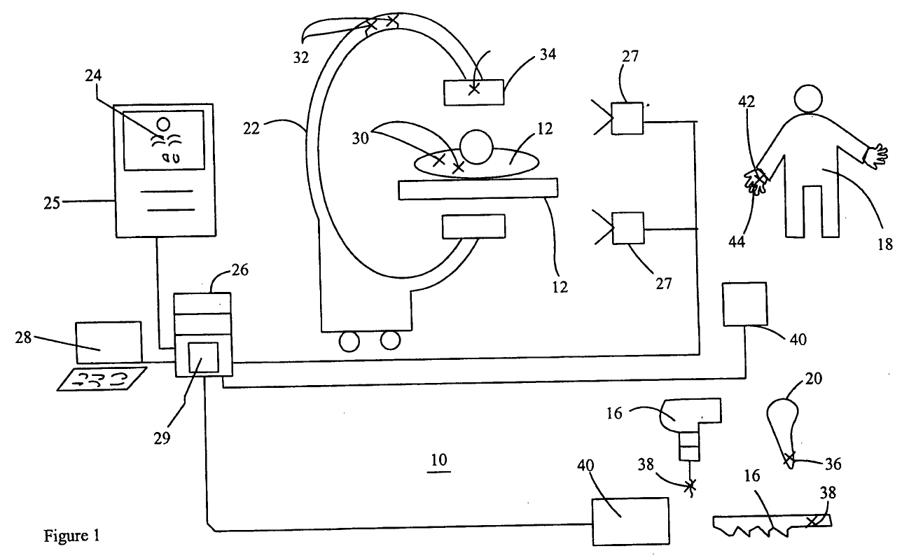

Referring to FIG. 1, there is shown a computer assisted surgery system 10 for performing open surgical procedures and minimal invasive surgical procedures on a patient 12 usually positioned horizontally on an operating table 14. Open surgical procedures include hip, knee and trauma surgeries, however computer assistance can facilitate minimal invasive approaches by providing valuable imaging information of normally hidden anatomy. Minimal invasive surgical procedures include keyhole approaches augmented by calibrated image information which reduce hospital stay and cost and greatly improve patient 12 morbidity and suffering. Such surgical procedures require a plurality of instruments 16, such as drills, saws and raspers. The system 10 assists and guides a user 18, such as a medical practitioner, to perform surgical procedures, such as to place implants 20 using the instruments 16, by providing the user 18 with positioning and orientation of the instruments 16 and implants 20 with relation to the patient's 12 anatomical region of the operation, such as the hip area.

As a general overview, the system 10 is used to assist the surgeon in performing an operation by acquiring and displaying an image of the patent. Subsequent movement of the patient and instruments is tracked and displayed on the image. Images of a selection of implants are stored by the system and may be called to be superimposed on the image. The surgical procedures may be planned using the images of the patient and instruments and implants and stored as a series of sequential tasks referred to defined datums, such as inclination or position. Gestures of the surgeon may be used in the planning stage to call the image of the instruments and in the procedure to increment the planned tasks.

Referring to FIG. 1, the system 10 includes an imaging device 22 for providing medical images 24, such as X-ray, fluoroscopic, computed tomography (CT), magnetic resonance imaging of the patient's 12 anatomical region of the operation and the relative location of the instruments 16 and implants 20. Generally, a C-arm, which provides X-ray and fluoroscopic images 24, is used as the imaging device 22. The C-arm can be positioned in the most convenient location for the procedure being carried out, while allowing the user 18, the maximum possible space in which to work so that the procedures can be freely executed. The C-arm 22 features movement about or along three axes, so that the patient 12 can be easily approached from any direction. The C-arm 22 includes an X-ray source 21, an X-ray detector 23 and imaging software that converts the output of the detector into a format that can be imaged on display screen 25 for displaying the images 24 to the user 18.

Radiation exposure is a necessary part of any procedure for obtaining an image to assist in calculating the proper angle of the instruments 16 and implants 20, however, radiation exposure is considered to be a hazard, an exposure to the user 18 as well as the patient 12 during orthopaedic procedures using fluoroscopy is a universal concern. Consequently, a reduction in the amount of radiation exposure is highly desirable. Typically, the images 24 are acquired during pre-planning and stored in a image memory 29 on a computing device 26 coupled to the C-arm 22. As will be explained further below, the acquired images are referenced to a 3D coordinate framework. This may be done automatically by referencing the image to the framework when acquiring the image or manually by formatting the image to contain fiducials, either inherent from the imaged structure or added in the form of an opaque marker to permit registration between the images and patients. Generally, the computing device 26 is contained within a housing and includes input/output interfaces such as graphical user interface display 28 and input means such as mouse and a keyboard.

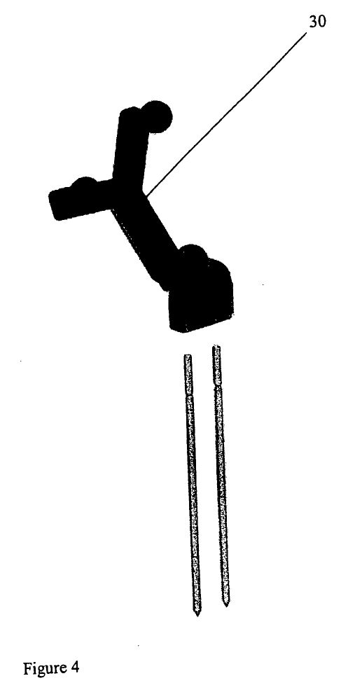

To facilitate the performance of the operation, the position and orientation of the operative instruments 16 and implants 20 is displayed on the images 24 by monitoring the relative positions of the patient 12, instruments 16 and implants 20. For this purpose, movement of the patient 12 is monitored by a plurality of positional sensors or patient trackers 30 as illustrated in FIG. 4 attached to the patient 12 to report the location of orientation of the patient 12's anatomy in a 3-D space. One example of the position sensor is a passive optical sensor, by NDI Polaris, Waterloo, Ontario, that allows real-time tracking of its trackers in three-dimensional space using an infrared-based camera tracking 27. Therefore, the patient trackers 30 report these coordinates to an application program 32 of the computing device 26. Each patient tracker 30 is fixed relative to the operative site, and a plurality of patient trackers 30 are used to accommodate relative movement between various parts of the patient's 12 anatomy. For minimal invasive surgery, the patient trackers 30 used can have minimal access for attachment to the patient 12.

To enable registration between the patient and the image during the procedure, position sensors 32 are placed in distinctive patterns on the C-arm 22. A tracking shield and grid 34, such as fiducial grid 34, are fitted onto the image intensifier of the C-arm 22. The grid 34 contains a set of markers 36 that are visible in images 24, and allow the image 24 projection to be determined accurately. The position sensors 36 with the tracked fiducial grid 32 are used to calibrate and/or register medical images 26 by fixing the position of the grid relative to the patient trackers 30 at the time the image is acquired.

The system 10 also includes hardware and electronics used to synchronize the moment of images 24 acquisition to the tracked position of the patient 12 and/or imaging device 22. The systems 10 also includes electronics to communicate signals from the position sensors 30, 36,38 or communicate measurements or information to the computing device 26 or electronics to the computing device 26 or other part of the system 10.

The instruments 16 also include positional sensors 38, or instrument trackers that provide an unambiguous position and orientation of the instruments. This allows the movement of the instruments 16 to be tracked virtually represented on the images 26 in the application program while performing the procedure. Some instruments 16 are designed specifically for the navigation system 10, while existing orthopedic instruments 16 can be adapted to work with the navigation system 10 by rigidly attaching trackers 34 to some part of the instrument 16 so that they become visible to the camera. By virtue of a tracker attached to an instrument, the position and trajectory of the instrument in the 3D coordinate system, and therefore relative to the patient can be determined. The trackers 38 fit onto the instruments 16 in a reproducible location so that their relation can be pre-calibrated. Verification that this attachment has not changed is provided with a verification device. Such a verification device contains “docking stations” where the instruments 16 can be positioned repeatedly relative to fixed locations and orientations. Existing instruments can be adapted by securing abutments on to the surgical instruments in a known position/orientation with respect to the instrument's axes. The calibration can be done by registering the position when in the docking station with a calibration device and storing and associating this calibration information with the particular docking station.

Alternatively, the docking station could be mechanically designed such that it has a unique position for the instrument in the docking station and such that the calibration information could be determined through the known details and configuration of the instrument.

Accordingly, the instrument and its associated tracker, can be removed from the docking station and its position monitored.

Similarly, the implants 20 include trackers 36 which may be integrated in to the implant or detachably secured so as to be disposable after insertion. The trackers 36 provide positional information of the implant 20 detectable by the system 10. The devices 36 transmit a signal to the tracking system 27 regarding their identity and position. The trackers on the devices 36 may include embedded electronics for measurement, computing and display allowing them to calculate and display values to the system 10 or directly to the user and may include a user-activated switch.

Images 26 of the patient 12 are taken and landmarks identified after patient trackers are rigidly mounted and before surgical patient positioning and draping on a surgical table 14. The images 26 are manually or automatically “registered” or “calibrated” by identification of the landmarks on both the patient and image. Since the images 26 are registered and saved on the computer readable medium of the computing device with respect to the tracker location, no more imaging may be required, unless required during the procedure. Therefore there is minimal radiation exposure to the user 18.

To assist in the planning of the procedure, the computing device of the system 10 includes stored images of implants and instruments compatible to the imaging system utilised. With an X-ray device, the images are generated by an algorithm for generating a 2D projection of instruments 16 and implants 22 onto 2D X-ray images 24. This involves algorithms that take the 3D CAD information and generate a 2D template that resembles templates that surgeons 18 are familiar with for planning. For example, the projection of the 3D femoral stem and acetabular cup model onto the X-ray is performed using a contour-projection method that produces the dynamic template that has some characteristics similar to the standard 2D templates used by surgeons 28, and therefore is more intuitive.

The “dynamic 2D template” from the 3D model provides both the exact magnification and orientation of the planned implant on the acquired image to provide an intuitive visual interface. A 2D template generation algorithm uses the 3D geometry of the implant, and 3D-2D processing to generate a projection of the template onto the calibrated X-ray image. The 2D template has some characteristics similar to those provided by implant manufacturers to orthopaedic surgeons for planning on planar X-ray films. The application program 32 allows the user to maneuver the virtual images of prosthetic components or implants until the optimum position is obtained. The surgeon can dynamically change the size of component among those available until the optimum configuration is obtained.

To facilitate the actual procedure, the system 10 also automatically detects implant and/or instrument models, by reading the bar codes carried by the implants. The system 10 includes a bar code reader that automatically or semi-automatically recognizes a cooled opto-reflecting bar code on an implant 20 package by bringing it in the vicinity of a bar code reader of the system 10. The implants are loaded into the system 10 and potentially automatically registered as a “used inventory” item. This information is used for the purposes of inventory control within a software package that could be connected to the supplier's inventory control system that could use this information to remotely track supplier and also replenished when a system 10 indicates that it has been used. Each of the implants carries trackers that are used to determine the orientation and position relative to the patient and display that on the display 28 as an overlay of the patient image.

It is recognized that other active/passive tracking systems could be used, if desired. The tracking system 27 can be, but is not limited to optical, magnetic, ultrasound, etc. Could also include hardware, electronics or internet connections that are used for purposes, such as remote diagnostics, training, service, maintenance and software upgrades. Other tracking means electrically energizeable emitters, reflective markers, magnetic sensors or other locating means.

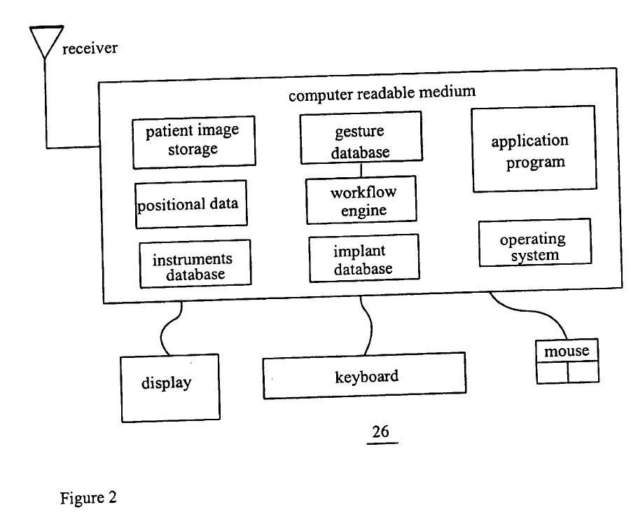

Each surgical procedure includes a series of steps such that there is a workflow associated with each procedure. Typically, these steps or tasks are completed in sequence. For each procedure the workflow is recorded by a workflow engine 38 in FIG. 2 coupled to the application program 32. Thus, the system 10 can guide the user 18 by prompting the user 18 to perform the task of the workflow or the user 18 directs the workflow to be followed by the system 10 by recognizing the tracked instruments 16 as chosen by the user 18. Generally, a combination of both guided workflows are possible in any given procedure. Thus, the user 18 can trigger an action for a specific workflow task. When the system 10 detects that a given task of the procedure has been invoked, it displays the required information for that procedure, pertinent measurements, and/or medical images 24. The system 10 also automatically completes user 18 input fields to specify certain information or actions. The guide also alerts the user 18 if a step of the workflow has been by-passed.

The tasks of the procedure are invoked by the user 18 interacting with the system 10 via an interface sub-system 40. The user 18 includes position sensors 42 or user trackers, typically mounted on the user's 18 hand. These sensors 42 provide tracking of user's 18 position and orientation. Generally, a hand input device 44 with attached tracker 42 or an electroresistive sensing glove is used to report the flexion and abduction of each of the fingers, along with wrist motion. Thus, each task of the workflow is associated with hand gestures, the paradigm being gesturally-based hand gestures to indicate the desired operation.

Hand gestures may also be used during planning. For example, the user 18 could make the “drill” gesture and the corresponding image, i.e. a virtual drill is called from the instrument image database and applied to the patient 12 data (hip) in the environment. Similarly, a sawing motion invokes the femoral proximal cut guidance mode, while a twisting motion invokes a reamer guidance mode and shows a rasp to invoke the leg length and anteversion guidance mode. Hand gestures may also be used during the surgical procedure to invoke iteration of the work flow steps or other action required.

Prior to the start of the procedure, a plurality of hand gestures are performed by the user 18, recorded by the computing device 22, and associated with a desired action and coupled to the pertinent images 24, measurement data and any other information specific to that workflow step. Therefore, if during the procedure, the user 18 performs any of the recorded gestures to invoke the desired actions of the workflow; the camera detects the hand motion gesture via the position sensors 42 and sends this information to the workflow engine for the appropriate action. Similarly, the system 10 is responsive to the signal provided by the individual instruments 16, and, responds to the appearance of the instruments in the field of vision to initiate actions in the work flow. The gestures may include a period of time in which an instrument is held stationary or may be combinations of gestures to invoke certain actions.

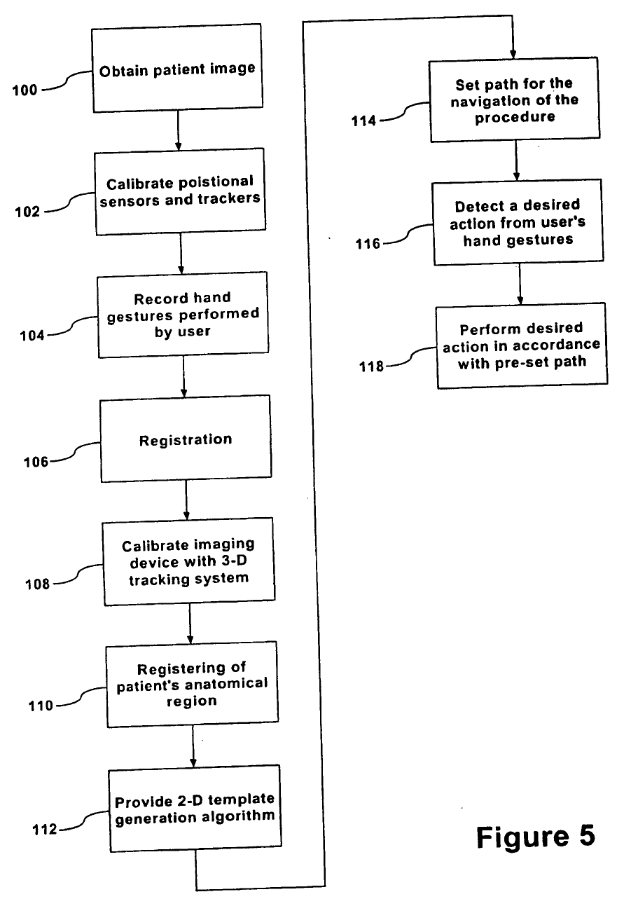

The steps for a typical method of a computer assisted surgery system 10 will now be described with the aid of a flowchart in FIG. 5.

Initially, patient trackers 30 are attached onto the patient 12 by suitably qualified medical personnel 18, and not necessarily by a surgeon 18. This attachment of trackers may be done while the patient 12 is under general anesthesia using local sterilization. The patient image is obtained using the C-arm 22 or similar imaging technique, so that either registration occurs automatically or characteristic markers or fiduciaries may be observed in the image. The markers may be readily recognized attributes of the anatomy being imaged, or may be opaque “buttons” that are placed on the patient.

The next step 102 involves calibrating the positional sensors or trackers on the instruments 16, implants 20 and a user's 18 hand in order to determine their position in a 3-dimensional space and their position in relation to each other. This is accomplished by insertion of the verification block that gives absolute position and orientation.

In the next step 104, a plurality of hand gestures are performed by the user 18 and recorded by the computing device 22. These hand gestures are associated with a desired action of the workflow protocol;

Registration is then performed if necessary between the image and patient by touching each fiduciary on the patient and image in succession. In this way, the image is registered in the 3D framework established by the cameras to that the relative movement between the instruments and patient can be displayed.

The next steps involves planning of the procedure. At step 10 the position of the patient's 12 anatomical region is registered. This step includes the sub-steps of tracking that patient's 12 anatomical region in space and numerically mapping it to a corresponding medical images 24 of that anatomy. This step is performed by locating some anatomical landmarks on the patient's 12 anatomical region with the 3D tracking system 27 and in the corresponding medical images 24 and calculating the transformation between 3D tracking and medical images 24 coordinate systems.

At step 112, the 2D templates of the instruments and implants generate a projection of the template onto the calibrated 2D X-ray images 24 in real time. The “dynamic 2D template” from the 3D model provides both the exact magnification and orientation of the planned implant with the intuitive visual interface. This step also includes generating a 2D projection of instruments 16 onto 2D X-ray images 24. The instruments 16 to be used on the patient 12 while performing the procedure are virtually represented on the images 24, and so are the implants. The 3D implant and instrument geometric models in combination are used with the registered medical images 24, and the generating 2D projections of that instrument and/or implant are updated dynamically in real-time as the implant/instrument is moved about in 3D space. Advantageously, the dynamic 2D projection is more intuitive and provides ease of use for a user 18. As the steps of the procedure are simulated, datums or references may be recorded on the image to assist in the subsequent procedure.

In the next 114, a path for the navigation of the procedure is set and the pertinent images 24 of the patient's 12 anatomical region are complied for presentation to the user 18 on a display. Thus the user 18 is presented with a series of workflow steps to be followed in order to perform the procedure.

After the planning stages, the procedure is started at step 116 by detecting a desired action from the user's hand gestures stored on said computer-readable medium; or from the positional information of a tracked instrument with respect to the tracking system 27 or other tracked device, or a combination of these two triggers;

The next step 118 involves performing the desired action in accordance with the pre-set path. However, the user 18 may deviate from the pre-set path or workflow steps in which case the system 10 alerts the user 18 of such an action. The system 10 provides visual, auditory or other sensory feedback to indicate when that the surgeon 18 is off the planned path. The 2D images 24 are updated, along with virtual representation of the implant 20 and instrument 16 positioning, and relevant measurements to suit the new user 18 defined path. After each step in the work flow, the user 18 increments the task list by gesturing or by selection of a different instrument. During the procedure, the references previously recorded provide feedback to the user 18 to correctly position and orientate the instruments and implants.

The method and system 10 for computer assisted surgery will now be described with regards to specific examples of hip and knee replacement. Hip replacement involves replacement of the hip joint by a prosthesis that contains two main components namely an acetabular and femoral component. The system 10 can be used to provide information on the optimization of implant component positioning of the acetabular component and/or the femoral component. The acetabular and femoral components are typically made of several parts, including for example inlays for friction surfaces, and these parts come in different sizes, thicknesses and lengths. The objective of this surgery is to help restore normal hip function which involves avoidance of impingement and proper leg length restoration and femoral anteversion setting.

In a total Hip or MIS Hip replacement guidance method, the clinical workflow starts with attachment of MIS ex-fix style patient trackers 30 in FIG. 5 on the patient's 12 back while under general anesthesia using local sterilization. The pins that fix the tracker to the underlying bone can be standard external fixation devices available on the market onto which a patient tracker is clamped. The user 18 interface of the system 10 prompts the user 18 to obtain the images 24 required for that surgery and associates the images 24 with the appropriate patient tracker 30. Once the images 24 have been acquired, the patient trackers 30 are maintained in a fixed position so that they cannot move relative to the corresponding underlying bone.

The system 10 presents images that are used to determine a plurality of measurements, such as the trans-epicondylar axis of the femur for femoral anteversion measurements. Femoral anteversion is defined by the angle between a plane defined by the trans-epicondylar axis and the long axis of the femur and the vector of the femoral neck To determine the orientation of the transcondylar axis of the femur, the C-arm 22 is aligned until the medial and lateral femoral condyles overlap in the sagittal view. This view is a known reference position of the femur that happens to pass through the transcondylar axis. The orientation of the X-ray image 24 is calculated by the system 10 and stored in the computer readable medium for later use. The transcondylar axis is one piece of the information used to calculate femoral anteversion.

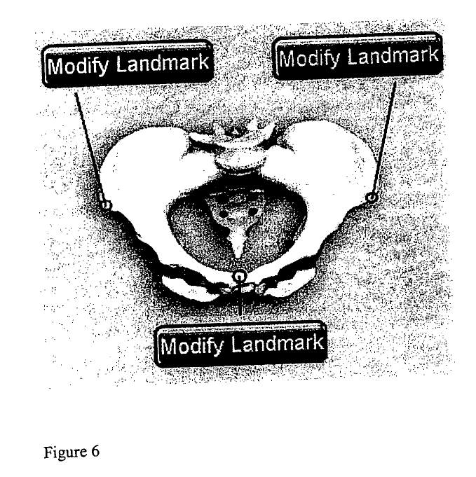

The system 10 includes intra-operative planning of the acetabular and femoral component positioning to help choose the right implant components, achieve the desired anteversion/inclination angle of the cup, anteversion and position of the femoral stem for restoration of patient 12 leg length and anteversion and to help avoid of hip impingement. Acetabular cup alignment is guided by identifying 3 landmarks on the pelvis that defines the pelvic co-ordinate system 10. These landmarks can be the left & right cases and pubis symphysis (See FIG. 6).

The position of the landmarks can be defined in a number of ways. One way is to use a single image 24 to refine the digitized landmark in the ante-posterior (AP) plane, as it is easier to obtain an AP image 24 of the hip than a lateral one due to X-ray attenuation through soft tissue. This involves moving the landmark within the plane of the image 24 without affecting its “depth” with respect to the X-ray direction of that image 24, as it is easier to obtain a single AP image 24 of the pelvis due to X-ray attenuation of the lateral image 24. The user 18 is made aware that the depth of the landmark must have been accurately defined through palpation or bi-planar digitization. Use of single X-ray images 24 can be used to ensure that the left and right axes are at the same “height” with respect to their respective pelvic crests and to ensure that the pubis symphysis landmark is well centered.

Alternatively, bi-planar reconstruction from two non-parallel images 24 of a given landmark can be used. This helps to minimize invasive localization of a landmark hidden beneath soft tissue or inaccessible due to patient 12 draping or positioning. The difference between modifying a landmark through bi-planar reconstruction and modifying the landmark position with the new single X-ray image 24 technique is that in bi-planar reconstruction, modification influences the landmark's position along an “x-ray beam” originating from the other image 24, whereas the single X-ray image 24 modification restricts landmark modification to the plane of that image 24.

The pelvic co-ordinate system 10 is used to calculate an anteversion/inclination angle of a cup positioner for desired cup placement. This can also be used to calculate and guide an acetabular reamer. The system 10 displays the anteversion/inclination angle to the user 18 along with a projection of the 3D cup position on X-ray images 24 of the hip. The details of calculations can be seen in FIG. 6.

For minimal invasive procedures, the system 10 provides navigation of a saw that is used to resect the femoral head. This step is performed before the acetabular cup guidance to gain access to the acetabulum. The system 10 displays the relevant C-arm 22 images 24 required for navigation of the saw and display the saw's position in real-time on those images 24. Guidance may be required for determining the height of the femoral cut. The system 10 then displays the relevant images 24 for femoral reaming and displays the femoral reamer. If the user 18 has selected an implant size at the beginning or earlier in the procedure, the system 10 displays the reamer corresponding to this implant size. Note that since reaming process starts with smaller reamers and works it's way up to the implant size, the virtual representation of the reamer will be larger than the actual reamer until the implant size is reached (for example for a size 12 implant, the surgeon 18 will start with a 8-9 mm reamer and work up in 1-2 mm increments in reamer size). This virtual representation allows the surgeon 18 to see if the selected implant size fits within the femoral canal. Secondly, it can help avoid the user 18 having to change the virtual representation on the UI for each reamer change which often occurs very quickly during surgery (time saving). The user 18 is able to change the reamer diameter manually if required.

The system 10 assists in guiding the orientation of the femoral reaming in order to avoid putting the stem in crooked or worse notching the intra-medullary canal, which can cause later femoral fracture. A virtual representation of the reamer and a virtual tip extension of the reamer are provided so the surgeon 18 can align the reamer visually on the X-ray images 24 to pass through the centre of the femoral canal. The system 10 allows the surgeon 18 to set a current reamer path as the target path. The system 10 provides a sound warning if subsequent reamers are not within a certain tolerance of this axis direction.

The femoral anteversion calculation is described below with the aid of FIG. 6:
where n_{probe} be a unit vector, pointing from the tip of the cup impactor towards the handle, and
n_{frontal}, nₐₓᵢₐₗ, and n_{saggital}, are unit vectors that are normal to the three orthogonal planes that form the pelvic co-ordinate system.
n_{frontal} be a unit vector, normal to the frontal plane of the patient 12, whose sense is from the posterior to the anterior of the patient 12.
nₐₓᵢₐₗ be a unit vector, normal to the axial plane of the patient 12, whose sense is from the inferior to the superior of the patient 12.
n_{saggital} be a unit vector, normal to the sagittal plane of the patient 12, whose sense is from patient 12 right to patient 12 left.
Let α represent the anteversion.
Let β represent the inclination.

$${v}_{probe\_frontal} = ( {n}_{probe} \cdot {n}_{axial} ) {n}_{axial} + ( {n}_{probe} \cdot {n}_{sagittal} ) {n}_{sagittal}$$

$${n}_{probe\_frontal} = {v}_{probe\_frontal} / \vert {v}_{probe\_frontal} \vert$$

$$α = 90 - {cos}^{- 1} ( {n}_{frontal} \cdot {n}_{probe} )$$

$$β = sign \times \{ 180 - {cos}^{- 1} ( {n}_{axial} \cdot {n}_{probe\_frontal} ) \}$$

where:
For a left hip ‘sign’ is positive unless n_{probe}_{_{—}}_{frontal}·n_{sagittal}<0.
For a right hip ‘sign’ is positive unless n_{probe}_{_{—}}_{frontal}·n_{sagittal}>0.

The system 10 also provides a technique for obtaining the trans-epicondylar axis of the femur. An accepted radiological reference of the femur is the X-ray view where the distal and posterior femoral condyles overlap. The direction of this view also happens to be the trans-epicondylar axis. The fluoro-based system 10 tracks the position of the image 24 intensifier to determine the central X-ray beam direction through C-arm 22 image calibration. The epicondylar axis is obtained by acquiring a C-arm 22 image that aligns the femoral condyles in the sagittal plane and recording the relative position of the C-arm 22 central X-ray beam with respect to the patient tracker.

Once these vectors are defined in the workflow, the system 10 will provide real-time update of femoral anteversion for a femoral rasp and femoral implant guides. A femoral rasp is an instrument inserted into the reamed femoral axis and used to rasp out the shape of the femoral implant. It is also possible to provide femoral anteversion measurements for other devices that may be used for anteversion positioning (for example the femoral osteotome). The system 10 also updates in real-time the effect of rasp or implant position on leg length. Leg Length is calculated in three steps. In the first step, before the hip is dislocated, the distance between a femoral tracker, T_{f}, and a pelvic tracker, Tₚ are obtained. Therefore, the initial distance, Lᵢ=(T_{f}−Tₚ)·nₐ.

The second step of the process involves calculating the new leg length fraction attributed to the acetabular cup position, L_{c}. Once the cup has been placed, the position of the cup impactor, Pᵢ, is stored. After the acetabular cup shell and liner have been selected, the exact location of the center of rotation along the impactor axis, P_{c} is obtained from the 3D models of the implants. The center of rotation is then projected onto the pelvic normal and relative to the pelvic tracker, and the length attributed by cup position, L_{c}=P_{c}·nₐ.

In the next step, the new leg length fraction attributed to the femoral stem position, Lₛ, is obtained. After selection of the desired stem and head implants, the precise location of the femoral head is obtained from the 3D models of the implants, Pₕ. As the femur is being rasped, the length is continuously calculated along the anatomical axis of the femur, V_{femur}, relative to the femoral tracker, T_{f} by monitoring the position of the reamer. The length attributed to stem position, Lₛ=Pₕ·V_{femur}.

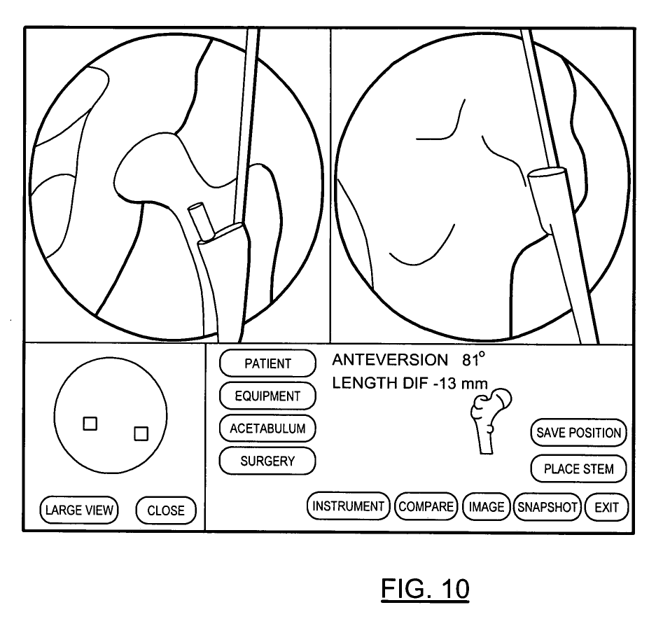

The implant models and components can be changed “on the fly” and the resulting effect on the above parameters displayed in real-time by the computer-implemented system 10. As indicated in FIG. 10, the application program implements algorithms which take into consideration changes in parameters such as component shape size and thickness to recalculate leg length and anteversion angles. Intra-operative planning may be important in hips or knees where bone quality is not well known until the patient 12 is open and changes in prosthesis size and shape may need to be performed intra-operatively. When a new component is chosen or when the surgeon 18 rasps further down into the femur than planned, due to poorer than expected bone quality for example, the system 10 will automatically generate updated leg length measurements and anteversion angles so that in situ decisions can be made. For example if the surgeon 18 has rasped too far into the femur, which would result in a leg length loss, the system 10 could be used to see if a larger sized femoral neck length or larger size femoral implant could be used to maintain the correct leg length.

The system 10 also calculates potential impingement in real-time between femoral and acetabular components based on the recorded acetabular cup position and the current femoral stem anteversion. Implant-implant impingement calculation is based on the fact that the artificial joint is a well-defined ball and socket joint. Knowing the acetabular component and femoral stem component geometry, one can calculate for which clinical angles impingement will occur. If impingement can occur within angles that the individual is expected to use, then the surgeon 18 is warned of potential impingement. Once the acetabular component has been set, the only remaining degree of freedom to avoid impingement is the femoral anteversion.

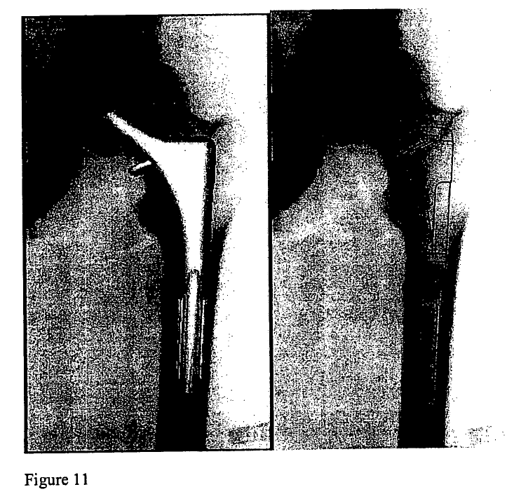

As mentioned above, the system 10 generates a 2D projection of implants onto 2D X-ray image 24 to provide the surgeon 18 with a more familiar representation, as shown in FIG. 11. The 2D projection model would be updated as the implant is rotated in 3D space.

The system 10 can also optionally record information such as the position of the femoral component of the implant or bony landmarks and use this information to determine acetabular cup alignment that minimizes the probability of implant impingement. This can help guide an exact match between acetabular and femoral anteversion for component alignment. The system 10 can help guide the femoral reamer that prepares a hole down the femoral long axis for femoral component placement to avoid what is termed femoral notching that can lead to subsequent femoral fracture. The system 10 provides information such as a virtual representation of the femoral reamer on one or more calibrated fluoroscopy views, and the surgeon 18 can optionally set a desired path on the image 24 or through the tracking system 27, and includes alerts indicative of the surgeon 18 straying from the planned path.

The system 10 guides the femoral rasp and provides femoral axis alignment information such as for the femoral reamer above. The chosen rasp position usually defines the anteversion angle of the femoral component (except for certain modular devices that allow setting of femoral anteversion independently). Femoral anteversion of the implant is calculated by the system 10 using information generated by a novel X-ray fluoroscopy-based technique and tracked rasp or implant position. It is known that an X-ray image 24 that superimposes the posterior condyles defines the trans-epicondylar axis orientation. If the fiducial calibration grid 34 is at a known orientation with respect to the X-ray plane in the tracking system 27 (either through design of the fiducial grid 34 or through tracking of both the fiducial grid 34 and the C-arm 22), the system 10 knows the image 24 orientation and hence the trans-epicondylar axis in the tracking co-ordinate system 10. The system 10 then can provide the surgeon 18 with real-time feedback on implant anteversion based on planned or actual implant position with respect to this trans-epicondylar axis. Alternative methods of obtaining the trans-epicondylar axis include direct digitization or functional rotation of the knee using the tracking device.

Proper femoral anteversion is typically important to help avoid impingement, as is the anteversion/inclination angle of the acetabular component. Since impingement occurs due to the relative orientation between the acetabular and femoral components, the system 10 optimizes femoral anteversion based on the acetabular component orientation if the latter was recorded by the tracking 27 as described above.

The effect of implant position on leg length, femoral anteversion and “impingement zone” is updated in real-time with the planned or actual implant position taking into account the chosen acetabular component position. Implant model and components can be changed “on the fly” and used by the surgeon 18 through and the resulting effect on the above parameters displayed in real-time.

The technology involves “intelligent instruments” that, in combination with the computer, “know what they are supposed to do” and guide the surgeon 18. The system 10 also follows the natural workflow of the surgery based on a priori knowledge of the surgical steps and automatic or semi-automatic detection of desired workflow steps. For example, the system 10 provides the required images 24 and functionality for the surgical step invoked by a gesture. Specific examples of gestures within the hip replacement surgery include picking up the cup positioner to provide the surgeon 18 with navigation of cup anteversion/inclination to within one degree (based on identification of the left & right axes and pubis symphysis landmarks), picking up the reamer and the rasp will also provides the appropriate images 24 and functionality, while picking up the saw will provide interface for location and establishment of the height that the femoral head will be cut. The surgeon 18 can skip certain steps and modify workflow flexibly by invoking gestures for a given step. The system 10 manages the inter-relationships between the different surgical steps such as storing data obtained at a certain step and prompting the user 18 to enter information required for certain.

Disposable components for a hip instrumentation set include a needle pointer, a saw tracker, an optional cup reamer tracker, a cup impactor tracker, a drill tracker (for femoral reamer tracking), a rasp handle tracker, a implant tracker, and a calibration block.

In another example, the system 10 is used for a uni-condylar knee replacement. The uni-knee system 10 can be used without any images 24 or with fluoro-imaging to identify the leg's mechanical axes. The system 10 allows definition of hip, knee and ankle center using palpation, center of rotation calculation or bi-planar reconstruction.

The leg varus/valgus is displayed in real-time to help choose a uni-compartmental correction or spacer. The surgeon 18 increases the spacer until the desired correction is achieved. Once the correction is achieved, the cutting jig is put into place for the femoral cut. The tibial cuts and femoral cuts can be planned “virtually” based on the recorded femoral cutting jig position before burring. In the case of a system 10 that uses a burr to prepare the bone, two new methods for guiding the burr are particularly beneficial. The first is a “free-hand” guide that tracks the burr. A cutting plane or curve is set by digitizing 3 or more points on the bone surface that span the region to be burred. The system 10 displays a color map representing the burr depth in that region and the color is initially all green. The desired burr depth is also set by the user. As the surgeon 18 burrs down, the color at that position on the colormap turns yellow, orange then red when the burr is within 1 mm of desired depth (black will indicate that burr has gone too far). The suggested workflow is to “borrow” burr holes at the limits of the area to be burred down to the red zone under computer guidance. The surgeon 18 then burrs in between these holes only checking the computer when he/she is unsure of the depth. The system 10 can also provide sound or vibration feedback to indicate burring depth.

A small local display or heads-up display can help the surgeon 18 concentrate on the local situs while burring. For the curved surface of the femur, the colormap represents the burr depth along a curve.

The second method presented is a passive burr-guide. The following is an example: a cutting jig has one to four base pins and holds a “burr-depth guide” that restricts burr depth to the curved (in femur) or flat (in tibia) implant. The position and orientation of this device is computer guided (for example by controlling height of burr guide on four posts that place it onto the bone). The burr is run along this burr guide to resect the required bone. As in the hip replacement procedure, the patient trackers 30 are positioned similarly.

The system 10 can also be linked to a pre-operative planning system in a novel manner. Pre-operative planning can be performed on 2D images 24 (from an X-ray) or in a 3D dataset (from a CT scan). These images 24 are first corrected for magnification and distortion if necessary. The implant templates or models are used to plan the surgery with respect to manually or automatically identified anatomical landmarks. The pre-operative plan can be registered to the intra-operative system 10 through a registration scheme such as corresponding landmarks in the pre and intra-operative images 24. Other surface and contour-based methods are also alternative registration methods. In the case of hip replacement, for example, the center of the femoral head and the femoral neck axis provide such landmarks that can be used for registration. Once these landmarks have been identified intra-operatively, the system 10 can position the planned implant position automatically, which saves time in surgery. The plan can be refined intra-operatively based on the particular situation, for example if bone quality is not as good as anticipated and a larger implant is required.

Although the invention has been described with reference to certain specific embodiments, various modifications thereof will be apparent to those skilled in the art without departing from the spirit and scope of the invention as outlined in the claims appended hereto.

1. A computer-implemented method for enhancing interaction between a user and a surgical computer assisted system, the method includes the steps of:
tracking a user's hand gestures with respect to a reference point;
registering a plurality of gesturally-based hand gestures and storing said gestures on a computer-readable medium;
associating each of said plurality of gesturally-based hand gestures with a desired action.
detecting a desired action by referencing said user's hand gestures stored on said computer-readable medium; and
performing the desired action.

2. A method of claim 1 wherein said hand gestures that is tracked by a tracking system through an instrument in the user's hand.

3. A method of claim 2 where said instrument includes a tracking module for providing identification of said instrument, said identification being recognizable by the tracking system.

4. A method of claim 3 wherein said instrument is associated with a desired action.

5. A method of claim 4 wherein said desired action is detected by analyzing timing information related to an amount of time the instrument is held in a certain position with respect to reference point.

6. The method of claim 1 wherein said a desired action can detected from combination of hand gestures.

7. A method of claim 1 wherein the hand gestures are associated with desired actions in a workflow of the user.

8. A method of claim 7 wherein said workflow includes a set of desired steps and when the system detects that a given step of the procedure has been invoked, it configures a user interface to provide the required information, such as measurements and/or medical images, and provides required functionality, such as user input fields to specify certain information or actions.

9. The method of claim 8 wherein the user can access any step of the procedure in any given order, and the system prompts the user to pass to a subsequent step if there is missing information.

10. A method of claim 1 where the system automatically detects an implant and/or instrument model.

11. A method of claim 10 wherein the said implant is encoded with the identifying information.

12. A method of claim 11 wherein said implant has a bar code readable by a bar code reader.

13. A method according to claim 12 wherein said implant package has a coded opto-reflecting bar-code information recognizable by the tracking system.

14. A method of claim 11 wherein said implant having been detected by the system is automatically registered as a “used inventory” item.

15. A method of a computer assisted surgery system to reduce user interaction by determining information for a surgical procedure from the orientation of a medical image from an imaging device.

16. A method of claim 15 wherein information regarding the orientation of the medical image is obtained by tracking said imaging device or tracking of a fiducial object associated with said imaging device and visible in the image.

17. A method of claim 16 wherein the orientation of the imaging device is used to determine the medio-lateral axis of a femur, said axis being used for biomechanical calculations of a leg.

18. A method of a computer assisted surgery system including the steps of attaching minimal invasive patient trackers to a patient, acquiring intraoperative images with respect to said patient trackers, registering said intraoperative images to those trackers and storing said intraoperative images on a computer readable medium, whereby said intraoperative images are used during surgery precluding the use of said imaging device during surgery.

19. A method of a computer assisted surgery system having the step of displaying a magnified virtual representation of a target instrument or implant size while smaller instruments or implants are being used.

20. A method of assisting a surgical procedure by obtaining an image of a portion of a patient on whom the procedure is to be performed,
registering said image and said patient in a three dimensional coordinate system,
monitoring movement of said patient in said three dimensional coordinate system,
monitoring movement of an instrument to be used in said procedure,
superimposing a representation of said instrument of said image and adjusting relative positions thereof on said image as the relative position of said instrument and said patient vary,
monitoring movement of an implant to be used in said procedure and supervising a representation thereof on said image and adjusting the relative position thereof on said image as the relative position of said implant and patient varies.

21. A method according to claim 20 wherein reference data is provided on said image to assist in positioning said instrument and/or implant.
